(19) United States
(12) Patent Application Publication
(10) Pub. No.: US 2024/0407834 A1
(43) Pub. Date: Dec. 12, 2024

(54) ELECTROSURGICAL DEVICE WITH DIRECTIONAL ELECTRODE

(71) Applicant: Boston Scientific Medical Device Limited, Galway (IE)

(72) Inventors: Christian Balkovec, Cambridge (CA); Eduardo Moriyama, Richmond (CA); Gareth Davies, Toronto (CA); Matthew Paul Joseph DiCicco, Toronto (CA)

(21) Appl. No.: 18/737,622

(22) Filed: Jun. 7, 2024

Related U.S. Application Data
(60) Provisional application No. 63/507,384, filed on Jun. 9, 2023.

(51) Int. Cl. A61B 18/14; A61B 17/00; A61B 18/00; A61B 18/12

(52) U.S. Cl. CPC A61B 18/148; A61B 2017/00867; A61B 2018/00083; A61B 2018/00607; A61B 2018/0091; A61B 2018/1253; A61B 2018/144

(57) ABSTRACT

An electrosurgical device is disclosed. The electrosurgical device includes a longitudinally extending shaft having a distal region. The shaft defines an axis. An electrode is disposed on the distal region. The electrode includes a longitudinal surface and a generally planar, exposed distalmost surface. The distalmost surface defines an edge. An insulator is disposed on the longitudinal surface to the edge. The insulator surrounds the electrode and is flush with the distalmost surface.

### CROSS REFERENCE TO RELATED APPLICATIONS

The present application claims priority to U.S. Provisional Patent Application Ser. No. 63/507,384, filed Jun. 9, 2023, the entire disclosure of which is incorporated herein by reference.

### TECHNICAL FIELD

The present disclosure relates to medical devices, systems and methods for use in surgical procedures. More specifically, this disclosure relates to electrosurgical devices, systems and methods that provide for cutting, coagulation, hemostasis, or sealing of bodily tissues including bone with an electrosurgical device.

### BACKGROUND

Electrosurgery includes such techniques as cutting, coagulation, hemostasis, and/or sealing of tissues with the aid of electrodes energized with a suitable power source such as an electrosurgical unit including a power generator. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. The electrosurgical devices are typically held by the surgeon and connected to the power source, such as the electrosurgical unit, via cabling.

Electrosurgical devices pass electrical energy through tissue between the electrodes to provide coagulation to control bleeding and hemostasis to seal tissue. Electrosurgical devices can also cut tissue with plasma formed on the electrode. Tissue that contacts the plasma experiences a rapid vaporization of cellular fluid to produce a cutting effect. Typically, cutting and coagulation are often performed with electrodes in the monopolar arrangement while hemostasis is performed with electrodes in the bipolar arrangement. Electrical signals can be applied to the electrodes either as a train of high frequency pulses or as a continuous signal typically in the radiofrequency (RF) range to perform the different techniques. The signals can include a variable set of parameters, such as power or voltage level, waveform parameters such as frequency, pulse duration, duty cycle, and other signal parameters that may be particularly apt or preferred for a given technique. For example, a surgeon could cut tissue using a first RF signal having a set of parameters to form plasma and control bleeding using a second RF signal having another set of parameters more preferred for coagulation. The surgeon could also use electrodes in a bipolar arrangement or a bipolar electrosurgical device for hemostatic sealing of the tissue that would employ additional RF signals having another set of parameters. Surgical parameters, including the parameters related to RF energy as well as fluid flow and other parameters are typically set and adjusted on the electrosurgical unit. The electrosurgical device, which includes the active electrode, are handheld and include activation switches, such as pushbuttons, to deliver the RF energy to the active electrode during surgery.

### SUMMARY

In an Example 1, an electrosurgical device, comprising: a longitudinally extending shaft having a distal region, the shaft defining an axis; an electrode disposed on the distal region, the electrode having a longitudinal surface and a generally planar, exposed distalmost surface, the distalmost surface defining an edge; and an insulator disposed on the longitudinal surface to the edge, the insulator surrounding the electrode and flush with the distalmost surface.

In an Example 2, the electrosurgical device of Example 1, wherein the exposed distalmost surface is generally perpendicular to the axis.

In an Example 3, the electrosurgical device of any of Examples 1 and 2, wherein the insulator includes a distal side flush with the distalmost surface.

In an Example 4, the electrosurgical device of any of Examples 1-3, wherein the insulator comprises a heat shrink polytetrafluoroethylene.

In an Example 5, the electrosurgical device of any of Examples 1-4, wherein the electrode comprises nitinol.

In an Example 6, the electrosurgical device of any of Examples 1-5, wherein the shaft includes a proximal region, and further comprising a handle coupled to the proximal region.

In an Example 7, the electrosurgical device of any of Examples 1-6, wherein the shaft is one of flexible and generally rigid.

In an Example 8, the electrosurgical device of Example 7, wherein the shaft is generally rigid and the electrosurgical device is a handheld device.

In an Example 9, the electrosurgical device of any of Examples 1-8, wherein the electrode is cylindrical.

In an Example 10, the electrosurgical device of any of Examples 1-9, wherein the electrode includes an outer puck disposed on a core mandrel.

In an Example 11, the electrosurgical device of Example 10, wherein shaft carries an electrical conductor, and the core mandrel is electrically coupled to the electrical conductor.

In an Example 12, the electrosurgical device of Example 11, wherein the core mandrel is integrally formed with the electrical conductor.

In an Example 13, the electrosurgical device of any of Examples 10-12, wherein the outer puck is threaded onto the core mandrel.

In an Example 14, the electrosurgical device of any of Examples 1-13, wherein the electrosurgical device is operably coupled to an electrosurgical unit and a pad dispersive electrode.

In an Example 15, the electrosurgical device of any of Examples 1-14, wherein the insulator is retractable to expose a portion of the longitudinal surface.

In an Example 16, an electrosurgical device, comprising: a longitudinally extending shaft having a distal region, the shaft defining an axis; an electrode disposed on the distal region, the electrode having a longitudinal surface and a generally planar, exposed distalmost surface, the distalmost surface defining an edge; and an insulator disposed on the longitudinal surface to the edge, the insulator surrounding the electrode and flush with the distalmost surface.

In an Example 17, the electrosurgical device of Example 16, wherein the insulator includes a distal side flush with the distalmost surface.

In an Example 18, the electrosurgical device of Example 16, wherein the insulator comprises a heat shrink polytetrafluoroethylene.

In an Example 19, the electrosurgical device of Example 16, wherein the electrode comprises nitinol.

In an Example 20, the electrosurgical device of Example 16, wherein the shaft includes a proximal region, and further comprising a handle coupled to the proximal region.

In an Example 21, the electrosurgical device of Example 16, wherein the shaft is one of flexible and generally rigid.

In an Example 22, the electrosurgical device of Example 21, wherein the shaft is generally rigid and the electrosurgical device is a handheld device.

In an Example 23, the electrosurgical device of Example 16, wherein the electrode is cylindrical.

In an Example 24, the electrosurgical device of Example 16, wherein the electrode includes an outer puck disposed on a core mandrel.

In an Example 25, the electrosurgical device of Example 24, wherein shaft carries an electrical conductor, and the core mandrel is electrically coupled to the electrical conductor.

In an Example 26, the electrosurgical device of Example 25, wherein the core mandrel is integrally formed with the electrical conductor.

In an Example 27, the electrosurgical device of Example 24, wherein the outer puck is threaded onto the core mandrel.

In an Example 28, the electrosurgical device of Example 24, wherein the outer puck is welded onto the core mandrel.

In an Example 29, the electrosurgical device of Example 16, wherein the shaft is configured as a guidewire.

In an Example 30, an electrosurgical system, comprising: an electrosurgical unit configured to provide a source of radiofrequency energy; and an electrosurgical device operably coupled to the electrosurgical unit, the electrosurgical device comprising: a longitudinally extending shaft having a distal region, the shaft defining an axis; an electrode disposed on the distal region, the electrode configured to receive the source of radiofrequency energy from the electrosurgical unit, the electrode having a longitudinal surface and a generally planar, exposed distalmost surface, the distalmost surface defining an edge; and an insulator disposed on the longitudinal surface to the edge, the insulator surrounding the electrode and flush with the distalmost surface.

In an Example 31, the electrosurgical system of Example 30, wherein the electrosurgical device is configurable in a plurality of functions including a cut function and a coagulation function.

In an Example 32, the electrosurgical system of Example 30, wherein the electrosurgical device is configurable in a monopolar mode.

In an Example 33, the electrosurgical system of Example 30, and further comprising a pump couplable to source of fluid via delivery tubing, the electrosurgical unit configured to operate the pump, and the delivery tubing operably coupled to electrosurgical device.

In an Example 34, an electrosurgical device, comprising: a longitudinally extending shaft having a distal region, the shaft defining an axis; an electrode disposed on the distal region, the electrode having a longitudinal surface and a generally planar, exposed distalmost surface, the distalmost surface defining an edge; and an insulator disposed on the longitudinal surface to the edge, the insulator surrounding the electrode and flush with the distalmost surface; wherein the insulator is transitionable to a retracted position wherein a portion of the longitudinal surface is exposed.

In an Example 35, the electrosurgical device of Example 34, wherein the insulator is slidable along the longitudinal surface.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, the example clinical setting having an example electrosurgical system including an example electrosurgical unit in combination with a fluid source and an electrosurgical device.

FIG. 2 is a schematic diagram illustrating an example electrosurgical device that can be used with the example electrosurgical system. of FIG. 1.

FIG. 3A is a schematic diagram illustrating from a perspective view a feature of an example of the electrosurgical device of FIG. 2.

FIG. 3B is a schematic diagram illustrating from a sectioned perspective view a feature of another example of the electrosurgical device of FIG. 2

FIG. 4 is a schematic diagram illustrating from a side view an example electrosurgical device of FIG. 2.

FIG. 5A is a schematic diagram illustrating from a side view a first position of another example electrosurgical device of FIG. 2.

FIG. 5B is a schematic diagram illustrating from a side view a second position of the another example electrosurgical device of FIG. 2

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

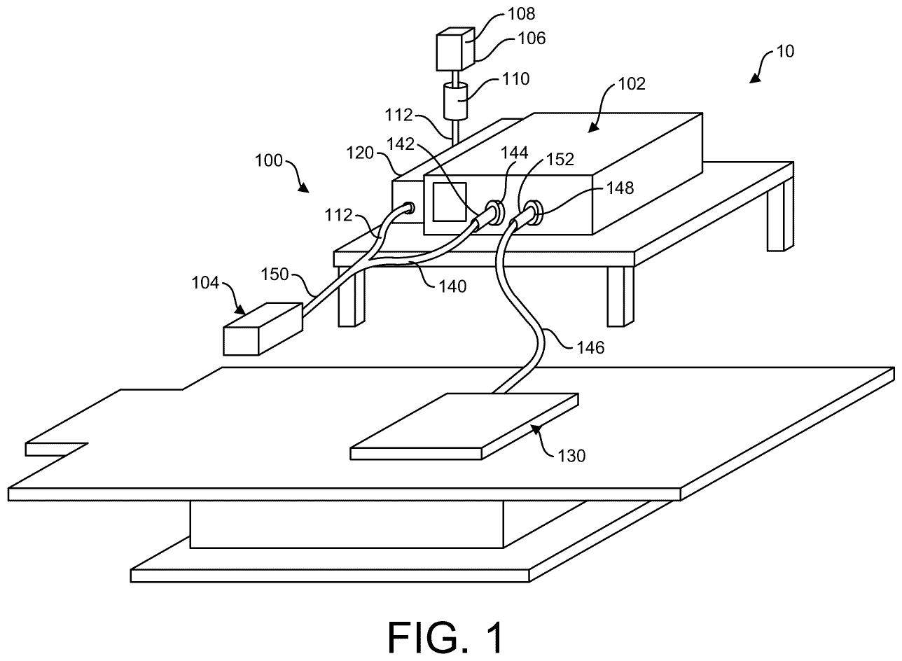

FIG. 1 illustrates an example of a clinical setting 10 for treating a patient. Clinical setting 10 comprises a system 100 that includes an electrosurgical unit 102 in combination with an example electrosurgical device 104. The device 104, in some embodiments, is configurable for use in cutting and sealing, including electrocautery, coagulation in a monopolar mode. In some embodiments, device 104 is used in combination with a fluid source 106, such as for irrigation or for other electrical surgical procedures.

Fluid source 106 may comprise a bag of fluid from which fluid 108 may flow through a drip chamber 110, to delivery tubing 112 and to handheld electrosurgical device 104. In one example, the fluid 108 includes saline and can include physiologic saline such as sodium chloride (NaCl) 0.9% weight/volume solution. Saline is an electrically conductive fluid, and other suitable electrically conductive fluids can be used. In other examples, the fluid may include a nonconductive fluid, such as deionized water, which may still provide advantages over using no fluid and may support cooling of portions of electrosurgical device 104 and tissue or reducing the occurrence of tissue sticking to the electrosurgical device 104.

The fluid delivery tubing 112 in the illustrated embodiment passes through pump 120 to convey fluid to the electrosurgical device 104 and control fluid flow. Pump 120 in one example is a peristaltic pump such as a rotary peristaltic pump or a linear peristaltic pump. A peristaltic pump can convey the fluid through the delivery tubing 112 by way of intermittent forces placed on the external surface of the delivery tubing. Peristaltic pumps are often applied during use of the electrosurgical device 104 because the mechanical elements of the pump places forces on the external surface of the delivery tubing and do not come into direct contact with the fluid, which can reduce the likelihood of fluid contamination. Other embodiments of system 100 do not include a pump, and fluid is provided to the electrosurgical device 104 via gravity, or, in some embodiments, not provided to the electrosurgical device 104.

Embodiments of the electrosurgical unit 102 can provide monopolar, bipolar, or both monopolar and bipolar radio-frequency (RF) power output to a specified electrosurgical instrument such as electrosurgical device 104 or a plurality or suite of electrosurgical instruments applied in the clinical setting 10. In one example, the electrosurgical unit 102 can be used for delivery of RF energy to instruments indicated for cutting and coagulation of soft tissue and for delivery of RF energy concurrent with fluid to instruments indicated for hemostatic sealing and coagulation of soft tissue and bone. In one example, the electrosurgical unit 102 is capable of simultaneously powering specified monopolar and bipolar electrosurgical instruments but may include a lock out feature preventing both monopolar and bipolar output from being simultaneously activated.

During monopolar operation of electrosurgical device 104, a first electrode, often referred to as the active electrode, is provided with electrosurgical device 104 while a second electrode 130, often referred to as the indifferent or neutral electrode, is provided in the form of a ground pad dispersive electrode located on the patient. For example, the ground pad dispersive electrode 130 is typically on the back, buttocks, upper leg, or other suitable anatomical location during surgery. In such a configuration, the ground pad dispersive electrode 130 is often referred to as a patient return electrode. An electrical circuit of RF energy is formed between the active electrode and the ground pad dispersive electrode through the patient.

The electrosurgical device 104 in the example is connected to electrosurgical unit 102 via cable 140. Cable 140 includes plug 142 that connect with receptacles 144 on the electrosurgical unit 102. In one example, a receptacle can correspond with an active electrode receptacle and one or more receptacles can correspond with controls on the electrosurgical device 104. Still further, a receptacle can correspond with a return electrode receptacle for devices that can be operated in a bipolar mode. If the electrosurgical unit 10 may be used in monopolar mode, an additional cable 146 connects the ground pad electrode 130 with plug 152 to a ground pad receptacle 148 of the electrosurgical unit 102. In some examples, delivery tubing 112 and cable 140 are combined to form a single cable 150.

The features of electrosurgical unit 102 described are for illustration, and the electrosurgical units suitable for use with device 104 may include some, all, or other features than those described below. In one example, the electrosurgical unit 102 is capable of operating in monopolar and bipolar modes as well as multiple functions within a mode such as a monopolar cutting function and a monopolar coagulation function. In some examples, a monopolar device is capable of performing a monopolar hemostasis or tissue sealing function. In the monopolar cutting function, monopolar RF energy is provided to the device 104 at a first power level and/or a first waveform (collectively first RF energy setting). For example, RF energy for a cut function may be provided at a relatively low voltage and a continuous current (100% on, or 100% duty cycle). Nominal impedance can range between 300 to 1000 ohms for the cutting function. At a power setting of 90 Watts for cutting, voltage can range from approximately 164 to 300 volts root mean square (RMS). In the monopolar coagulation function, monopolar RF is energy is provided to the electrode at a second power level and/or second waveform (collectively second or coagulation RF energy setting) that is different than at least one of the first power level or the first waveform. For example, RF energy for a coagulation function may be provided at a relatively higher voltage than the cut voltage and with a pulsed current, such as 1% to 6% on and 99% to 94% off, respectively (or 1% to 6% duty cycle). Other duty cycles are contemplated. The electrosurgical unit 102 in the bipolar mode may provide bipolar RF energy at a third power level and/or third waveform (collectively third RF energy setting) to a bipolar device along with a fluid for a (generally low voltage) hemostasis or tissue sealing function that may the same as or different than the cutting and coagulation RF settings provided to the device 104 for the cut function or the coagulation function. In one example, hemostatic sealing energy can be provided with a continuous current (100% duty cycle). Nominal impedance can range between 100 to 400 ohms for the hemostatic sealing function. At a power setting of 90 Watts for hemostatic sealing, voltage can range from approximately 95 to 200 volts RMS.

In one example, the unit 102 provides RF energy to the active electrode as a signal having a frequency in the range of 100 KHz to 10 MHz. Typically this energy is applied in the form of bursts of pulses. Each burst typically has a duration in the range of 10 microseconds to 1 millisecond. The individual pulses in each burst typically each have a duration of 0.1 to 10 microseconds with an interval between pulses of 0.1 to 10 microseconds. The actual pulses are often sinusoidal or square waves and bi-phasic, that is alternating positive and negative amplitudes.

The electrical surgical unit 102 includes a power switch to turn the unit on and off and an RF power setting display to display the RF power supplied to the electrosurgical device 104. The power setting display can display the RF power setting numerically in a selected unit such as watts.

The example electrosurgical unit 102 includes an RF power selector comprising RF power setting switches that are used to select or adjust the RF power setting. A user can push one power setting switch to increase the RF power setting and push the other power setting switch to decrease the RF power setting. In one example, power setting switches are membrane switches, soft keys, or as part of a touchscreen. In another example, the electrosurgical unit may include more than one power selectors such as a power selector for monopolar power selection and a power selector for bipolar power selection.

In one example, the electrosurgical unit 102 provides the power to the device 104, but the actual power level delivered to the electrosurgical device 104 can be selected via controls on the electrosurgical device 104 rather than controls on the electrosurgical unit 102. In another example, the electrosurgical unit 102 can be programmed to provide power levels within a selected range of power, and the electrosurgical device 104 is used to select an output power level within the preprogrammed range. For instance, the unit 104 can be programmed to provide monopolar energy for a cut function in a first range of power settings. The unit 102 can be programmed to provide monopolar energy for a coagulation function in a second range of power settings, which second range may be the same as, different than, or overlap the first range. The user may then select the function and adjust the power setting within the range using controls on the device 104 rather than using controls on the unit 102. Other examples of controlling power setting with controls on the device 104 rather than with controls on the unit 102 are contemplated.

The example electrosurgical unit 102 can also include fluid flow rate setting display and flow rate setting selector. The display can include indicator lights, and the flow rate selector can include switches. Pushing one of the flow rate switches selects a fluid flow rate, which is than indicated in display. While not being bound to a particular theory, the relationship between the variables of fluid flow rate (such as in units of cubic centimeters per minute (cc/min)) and RF power setting (such as in units of watts) can be configured to inhibit undesired effects such as tissue desiccation, electrode sticking, smoke production, char formation, and other effects while not providing a fluid flow rate at a corresponding RF power setting not so great as to disperse too much electricity and or overly cool the tissue at the electrode/tissue interface. Electrosurgical unit 102 is configured to increase the fluid flow rate generally linearly with an increasing RF power setting for each of the three fluid flow rate settings of low, medium, and high.

Electrosurgical unit 102 can be configured to include control of the pump 120. In this example, the speed of the pump 120, and the fluid throughput, can be predetermined based on input variables such as the RF power setting and the fluid flow rate setting. In one example, the pump 120 can be integrated with the electrosurgical unit 102.

In one example, the electrosurgical unit via pump provides the fluid 108 to the device 104, but the actual rate of fluid flow delivered to the electrosurgical device 104 is selected via controls on the electrosurgical device 104 rather than controls on the electrosurgical unit 102. In another example, the electrosurgical unit 102 can be programmed to provide fluid flow rate within a selected range of rates of flow, and the electrosurgical device 104 is used to select fluid flow rate within the programmed range. For instance, the unit 102 can be programmed to provide fluid flow rate for monopolar operation in a first range of fluid flow settings. The unit 102 can be programmed to provide fluid flow for bipolar operation in a second range of fluid flow rate settings, which second range may be the same as, different than, or overlap the first range. The user may then select the mode and adjust the fluid flow rate within the range using controls on the device 104 rather than using controls on the unit 102. Other examples of controlling fluid flow with controls on the device 104 rather than with controls on the unit 102 are contemplated.

While electrosurgical surgical device 104 is described with reference to electrosurgical unit 102 and other elements of system 100, it should be understood the description of the combination is for the purposes of illustrating system 10. It may be possible to use the electrosurgical device 104 in other systems or the electrosurgical unit 102 may be used with other electrosurgical devices.

Electrosurgical devices in electrosurgical system can be applied to cut or puncture tissue. For instance, punctures in tissues can provide access for medical tools used in various medical interventions. In one example, a pericardium layer of a patient can be punctured to provide for epicardial access, such as to create an access point to insert tools for epicardial ablation. In one technique, a goal is to puncture the epicardial layer without puncturing the underlying myocardium. Electrosurgical device can also be applied to remove accumulation of atheromatous material on the inner walls of vascular lumens, which results in atherosclerosis. In one technique, the goal is to puncture through the vascular occlusion directly in front of an electrosurgical device without affecting the vessel walls.

Typical electrosurgical devices include limitations that make difficult their application to efficient puncture techniques. For example, electrosurgical devices with domes or shaped electrodes often provide RF energy in about a three-dimensional surface, which can deliver the RF energy to unintended tissue. For example, domed or shaped electrodes tend to cut unintended regions of pericardium tissue bunched from an introducer device or guidewire in contact with the tissue. Further, domed or shaped electrodes tend to apply RF energy to vascular walls not directly in front of the electrosurgical device when treating occlusions. Electrodes formed into a pointed tip can improve the direction of energy but also risk unintended mechanical cutting of tissue with the sharp points.

The disclosure relates to electrosurgical devices and electrosurgical systems that are configured to apply RF energy to tissue directly in front of the electrode, such as directly along an axis of the electrosurgical device, and not apply RF energy to tissue longitudinally along the electrode. Further, the electrode is formed so as to reduce the risk of mechanical cutting or puncture. Suitable uses for the electrosurgical device include in facilitating pericardial puncture to provide epicardial access and for the treatment of vascular occlusions. In one example, the electrosurgical device is manipulated to provide an electrode proximal to puncture intended along the axis of the electrode. Once in position, the electrode is activated with RF energy provided from the electrosurgical unit to form a puncture in the tissue or to vaporize or cut tissue or an occlusion. The tissue or occlusion directly in front of the electrode is affected, and tissue longitudinal to the electrode is not affected.

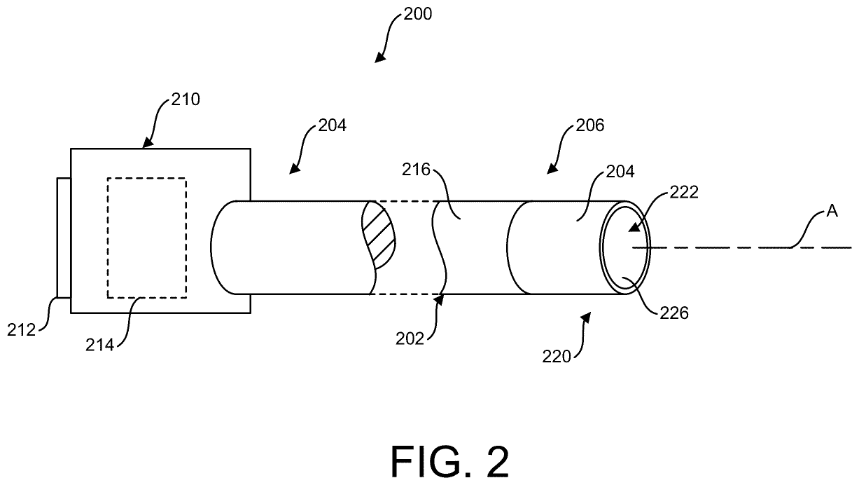

FIG. 2 illustrates an example electrosurgical device 200, which can correspond with electrosurgical device 104 and be used with electrosurgical unit in system 10. The example electrosurgical device 200 includes a longitudinally extending shaft 202 having a proximal region 204 and a distal region 206. The shaft 202 defines a longitudinal axis A. The proximal region 204 can be coupled to a handle 210 proximal to the shaft 202. A monopolar electrode assembly 220 is disposed on the distal region 204. The example electrosurgical device 200 can be configured as a handheld assembly in some examples, a catheterization tool such as a guidewire assembly for use with an introducer, or other suitable configuration. The electrosurgical device 200 can be operably coupled to the electrosurgical unit 102 and the ground pad dispersive electrode 130. In one example, the electrosurgical device 200 can receive a source of RF energy provided from the electrosurgical unit 102.

The handle 210 allows a user of the electrosurgical device 200 to hold the electrosurgical device 200 and manipulate or control the electrode assembly 220. In one example, the handle 210 includes an electrical connection 212 that can be coupled to cable 140 of system 10 such that the electrosurgical device 200 receives RF energy. In one example, the handle 210 can include a controller 214 that comprises one or more pushbuttons on the handle 210 in combination with circuitry such as a printed circuit board, or PCB, within the electrosurgical device 200 to provide binary activation (on/off) control for each function. For example, one button may be pressed to activate the monopolar electrode assembly 220 in, for instance, a cut function. In some examples, the electrosurgical device 200 can include a plurality of functions, and another button may be pressed to activate the monopolar assembly 220 in, for instance, a coagulation function. In some examples, still another button may be pressed to disperse fluid 108. Alternate configurations of the controller 214 and its activation are contemplated. In one example, the switches are remote from the handle 210 operably coupled to the handle 210, such as a foot switch or voice activation control.

The shaft 202 extends distally from the handle 210 and can include one or more elements forming a subassembly to be generally one or more of rigid, bendable, fixed-length, variable-length (including telescoping or having an axially-extendable or axially-retractable length) or other configuration. The shaft 202 carries one or more electrical conductors to a distal region 206 to the electrode assembly 220. The electrical conductors are operably coupled to the electrical connection 212 such as through controller 214. Electrical pathways within the handle 210 and shaft 202 can be formed as conductive arms, wires, traces, other conductive elements, and other electrical pathways formed from electrically conductive material such as metal and may comprise stainless steel, titanium, gold, silver, platinum or any other suitable material. The shaft 202 includes an outer cover 216, which can be formed of a biocompatible material. In one example, the outer cover 216 is formed from a thermoplastic elastomer (TPE). For example, the TPE can be a polyether block amide (PEBA) available under the trade designation PEBAX from Arkema, S.A., of Colombes, France, or under the trade designation VESTAMID E from Evonik Industries, AG, of Essen, Germany. In one example, the shaft 202 includes a fluid lumen extending into the handle 210 for fluidly coupling to delivery tubing 112 in cable 150. The fluid lumen includes an outlet port disposed on the electrode assembly 220 for selectively dispersing fluid 108.

The electrode assembly 220 is electrically coupled to the electrical connector 212 via the controller 214 and the electrical conductors within the shaft 202. The electrode assembly 220 includes a monopolar electrode 222 and an insulator 224. The electrode 222 is disposed on the distal region 206 and forms a distalmost surface 226 of the electrosurgical device 200. The distalmost surface 226 is generally perpendicular to the axis A. The insulator 224 is disposed on the longitudinal surface of the electrode 222 leaving exposed the distalmost surface 226. In one example, the insulator 224 is integrated with the outer cover 216 of the shaft 202. In another example, the insulator is a separate component.

The electrode assembly 220 is configured to direct the RF energy from the distalmost surface 226 to cut or puncture tissue with the distalmost surface 226, such as tissue directly in front of the electrode 222 along axis A. Tissue proximate the longitudinal surface of the electrode assembly, however, is not cut.

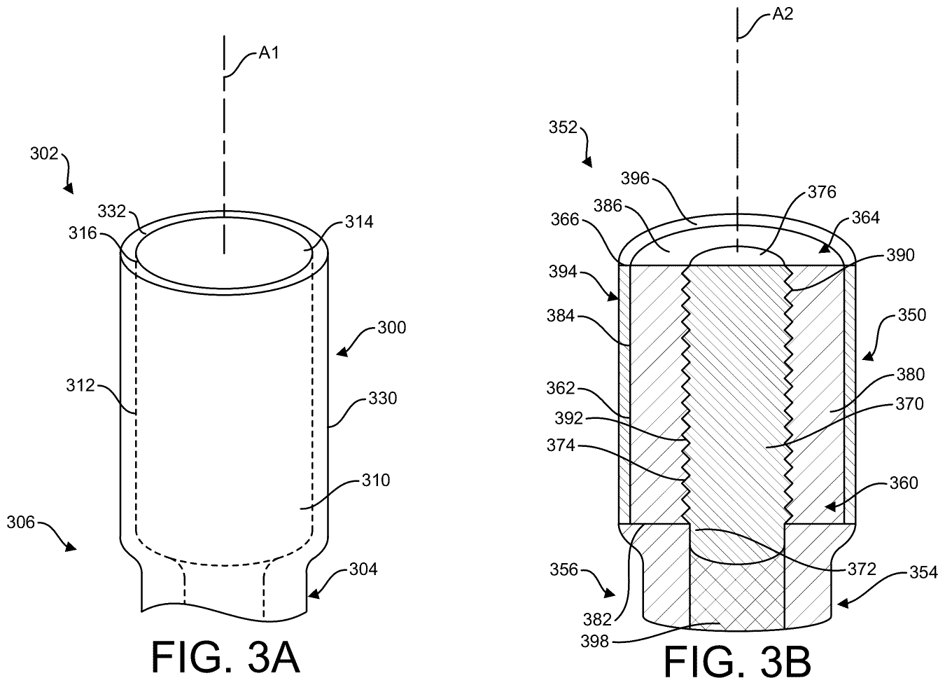

FIG. 3A illustrates an example electrode assembly 300 of an example electrosurgical device 302, the electrosurgical device 302 constructed in accordance with the electrosurgical device 200. The electrosurgical device 302 includes a longitudinally extending shaft 304 having a distal region 306, and the shaft 304 defines a longitudinal axis A1 in the example. The electrode assembly 300 includes an electrode 310 disposed on the distal region 306. The electrode 310 is electrically coupled to the electrical connector 212 and the controller 214 via the electrical conductors within the shaft 304. The electrode 310 is constructed from a conductive material, such as metal, and suitable to carry an RF signal from electrosurgical unit 102. The electrode 310 includes a longitudinal surface 312 and a generally planar, exposed distalmost surface 314. In the example, the distalmost surface 314 is generally perpendicular to the axis A1, although other configurations such as an angled and planar exposed distalmost surface. The longitudinal surface 312 surrounds the electrode 310. The distalmost surface 314 defines a distal edge 316, such as the union or ridge where the distalmost surface 314 meets the longitudinal surface 312. In the example, the electrode 310 is cylindrical such that it includes one longitudinal surface 312 and one distal edge 316 that extends around the distalmost surface 314. Another example can include a rectangular electrode that includes four sub-longitudinal surfaces and the distal edge can comprise four sub-edges that extend around a square or rectangular distalmost surface. Still other examples are contemplated.

The electrode assembly 300 also includes an insulator 330 disposed on the electrode 310. The insulator 330 is disposed on the longitudinal surface 312 to the edge 316 of the electrode 310. The insulator 330 is constructed from a suitable insulative material, such as a polymer. Examples insulative materials can include polytetrafluoroethylene (PTFE), polycarbonate (PC), polyoxymethylene (POM or acetal), or polyether ether ketone (PEEK). For example, the insulator 330 surrounds the electrode 310 on the longitudinal surface 312, or sub-longitudinal surfaces, and includes an insulator distal side 332 that is generally flush with the distalmost surface 314. For example, the insulator 330 extends distally all the way to the edge 316 such that the insulator distal side 332 coincides with the edge 316 and does not extend distally past the distalmost surface 314. In the example, no part of the longitudinal surface 312 of the electrode 310 is exposed, and all of the longitudinal surface 312 is covered with the insulator 330. Further, no conductive portion of the electrode assembly 300, shaft 304, or electrosurgical device 302 is exposed in the distal region 306 or portion of the electrosurgical device 302 that may contact the patient during normal operation is exposed. In the example, the only conductive element operably coupled to an RF signal exposed on the distal region 306 or electrosurgical device 302 is the distalmost surface 314. When the electrode 310 is activated, such as in a cut function, only the distalmost surface 314 applies RF energy to the patient. The distalmost surface 314 is used to puncture the tissue, while tissue proximate the longitudinal surface 312 is not cut.

FIG. 3B illustrates a sectioned view of another example electrode assembly 350 of an example electrosurgical device 352, the electrosurgical device 352 constructed in accordance with the electrosurgical device 200. The electrosurgical device 352 includes a longitudinally extending shaft 354 having a distal region 356, and the shaft 354 defines a longitudinal axis A2 in the example. The electrode assembly 350 includes an electrode 360 disposed on the distal region 356. The electrode 360 is electrically coupled to the electrical connector 212 and the controller 214 via the electrical conductors within the shaft 354. In the example electrode assembly 350, the electrode 360 includes components of a core mandrel 370 and an outer puck 380. The electrode 360 components of the core mandrel 370 and outer puck 380 in the example are each constructed from a conductive material, and suitable to carry an RF signal from electrosurgical unit 102.

The electrode 350 includes a longitudinal surface 362 and a generally planar, exposed distalmost surface 364. In the example, the distalmost surface 364 is generally perpendicular to the axis A2, although other configurations such as an angled and planar exposed distalmost surface are possible. The longitudinal surface 362 surrounds the electrode 350. The distalmost surface 364 defines a distal edge 366, such as the union or ridge where the distalmost surface 364 meets the longitudinal surface 362. In the example, the electrode 350 is cylindrical such that it includes one longitudinal surface 362 and one distal edge 366 that extends around the distalmost surface 364. Another example can include a rectangular electrode that includes four sub-longitudinal surfaces and the edge can comprise four sub-edges that extend around a square or rectangular distalmost surface. Still other examples are contemplated.

In the example, the core mandrel 370 forms an inner portion of the electrode 350, and the outer puck 380 forms an outer portion of the electrode 350. In one example, the core mandrel 370 is formed from a conductive alloy such as nickel titanium, or nitinol, and the outer puck is formed from platinum. The core mandrel 370 of the example is configured to be mechanically coupled to the electrical conductors in the shaft electrically coupled to the electrical connector 212 and the controller 214. The core mandrel 370 includes a proximal surface 372 that can be mechanically and electrically coupled to an electrical conductor 398 from the shaft 354. The core mandrel 370 in the example includes a longitudinally extending threaded surface 374 configured to mate with the outer puck 380 and a planar distal surface 376 opposite the proximal surface 372. In the example, the outer puck 380 is cylindrically shaped and includes a proximal surface 382, a longitudinal surface 384, and a planar distal surface 386. The longitudinal surface 384 and the planar distal surface 386 of the outer puck 380 defines the distal edge 366 of the electrode. The outer puck 380 also include an axial bore 390 in the proximal surface 382, such as a bore 390 extending through the puck 380. The bore 390 includes a threaded inner surface 392 of the outer puck 380 that can receive the threaded surface 374 of the core mandrel 370 and fit over the core mandrel 370 as illustrated. In one example, the outer puck 380 is laser welded to the core mandrel 370 or laser welded and threaded over the core mandrel 370. The core mandrel 370 and the outer puck 380 are connected together such that the planar distal surface 376 of the core mandrel 370 is flush with the planar distal surface 386 of the outer puck 380, in the example, to form the planar distalmost surface 364 of the electrode 350. Some examples, the core mandrel 370 can be axially recessed within the outer puck 380. The distal surface 376 of the core mandrel 370, however, does not extend distally from the distal surface 386 of the outer puck 380.

The electrode assembly 350 also includes an insulator 394 disposed on the electrode 360. The insulator 394 is disposed on the longitudinal surface 362 to the edge 366 of the electrode 360. The insulator 394 is constructed from a suitable insulative material, such as a polymer. Examples insulative materials can include a PTFE heat shrink. For example, the insulator 394 surrounds the electrode 360 on the longitudinal surface 362, or sub-longitudinal surfaces, and includes an insulator distal side 396 that is generally flush with the distalmost surface 364. In particular, the insulator 394 surrounds the longitudinal surface 386 of the outer puck 380. For example, the insulator 394 extends distally all the way to the edge 366 such that the insulator distal side 396 coincides with the edge 366 and does not extend distally past the distalmost surface 364. In the example, no part of the longitudinal surface 362 of the electrode 360 is exposed, and all of the longitudinal surface 362 is covered with the insulator 394. Further, no conductive portion of the electrode assembly 350, shaft 354, or electrosurgical device 352 is exposed in the distal region 356 or portion of the electrosurgical device 352 that may contact the patient during normal operation is exposed. In the example, the only conductive element operably coupled to an RF signal exposed on the distal region 356 or electrosurgical device 352 is the distalmost surface 364. When the electrode 360 is activated, only the distalmost surface 364 can apply RF energy to the patient.

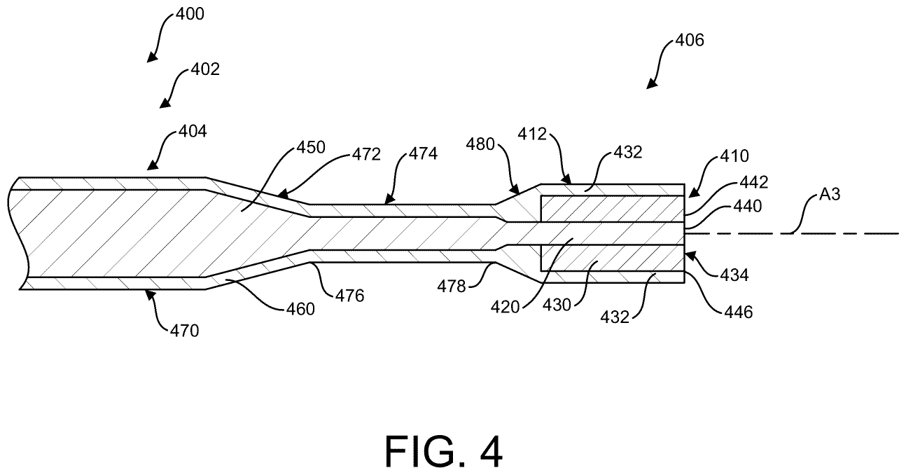

FIG. 4 illustrates a sectioned view of an electrosurgical device 400, including a distal region 402 of a shaft 404 and electrode assembly 406. The electrode assembly 406 is constructed in accordance with electrode assembly 350 and includes an electrode 410 and insulator 412. The electrode 410 of the example includes a core mandrel 420 and outer puck 430. In the longitudinally extending shaft 404 defines an axis A3. The electrode 410 is disposed on the distal region 402. The electrode 410 includes a longitudinal surface 432, such as a longitudinal surface of the outer puck 430. The electrode 410 also includes a generally planar, exposed distalmost surface 434, which comprises distal surfaces 440, 442 of the core mandrel 420 and outer puck 430, respectively. In the example, the generally planar, exposed distalmost surface 434 is generally perpendicular to the axis A3. The distalmost surface 434 and longitudinal surface 432 define an edge 446. The insulator 412 covers the electrode 410 along the longitudinal surface 432 to the edge 446 and is flush with the exposed distalmost surface 434. In one example, the only exposed conductive surface couplable to a source of RF energy is the exposed distalmost surface 434. When the electrode 410 is activated, only the distalmost surface 434 can apply RF energy to the patient.

For example, the shaft 404 includes a conductor 450 extending longitudinally along the axis A3 in the distal region 402 of the electrosurgical device 400. An insulative outer cover 460 is disposed on the conductor 450 along the axial length of the conductor 450 such that the cover 460 does not provide an exposed surface of the conductor 450. In the example, the conductor 450 integrally forms into the core mandrel 420 of the electrode 410. In one example, the conductor 450 and the core mandrel 420 are formed of the same material. In one example, the conductor 450 and core mandrel 420 are integrally formed from nitinol. In other examples, the conductor 450 is electrically coupled to the electrode, such as to the core mandrel 420. In this example, a mechanical coupling of the conductor 450 to the electrode 410 is covered with an insulative material such as the outer cover 460 and is not exposed. In one example, the outer cover 460 and insulator 412 are integrally formed together from PTFE heat shrink.

The example distal region 402 illustrates a taper toward the electrode assembly 406. In the example, a first portion 470 extends distally until a first taper, or narrowing taper 472. The narrowing taper 472 is characterized as a region in which the cross sectional diameter or area of the conductor 450 is reduced as the conductor 450 extends distally along axis A3. The narrowing taper 472 extends into a longitudinal portion 474. The longitudinal portion 474 extends axially between a proximal end 476 and a distal end 478, and the longitudinal portion 474 is characterized as a region in which the cross sectional diameter or area of the conductor 450 is generally constant as the conductor extends distally along axis A3. The distal end 478 of the longitudinal portion 474 can extend into a second taper, or widening taper 480. The conductor 450 at the widening taper 480 transitions into, or is coupled to, the electrode assembly 406.

In one example, the electrosurgical device is configured as a guidewire, and the electrode 410 is cylindrical. The distalmost surface 434 of the electrode 410 can include a diameter of approximately 0.026 inches, which is the outer diameter of the outer puck 430. The outer diameter of the core mandrel 420 on the distalmost surface 434 can be approximately 0.010 inches. The total outer diameter of the electrode assembly 406 including the outer diameter of the insulator 412 is approximately 0.035 inches and the axial length of the longitudinal side of the outer puck 430 is approximately 0.10 inches. The length of the shaft 404 can be approximately 180 centimeters, and the maximum outer diameter of the shaft 404 including the cover 460 can be approximately 0.018 inches. The axial length of the narrowing taper 472 can be approximately 2 centimeters, the axial length of the longitudinal portion 474 can be approximately 4.2 centimeters, and the axial length of the widening taper 480 can be approximately 0.1 centimeters. The outer diameter of the longitudinal portion 474 can be approximately 0.012 inches, and the outer diameter of the conductor 450 in the longitudinal portion 474 is approximately 0.010 inches, such as the same outer diameter of the core mandrel 420.

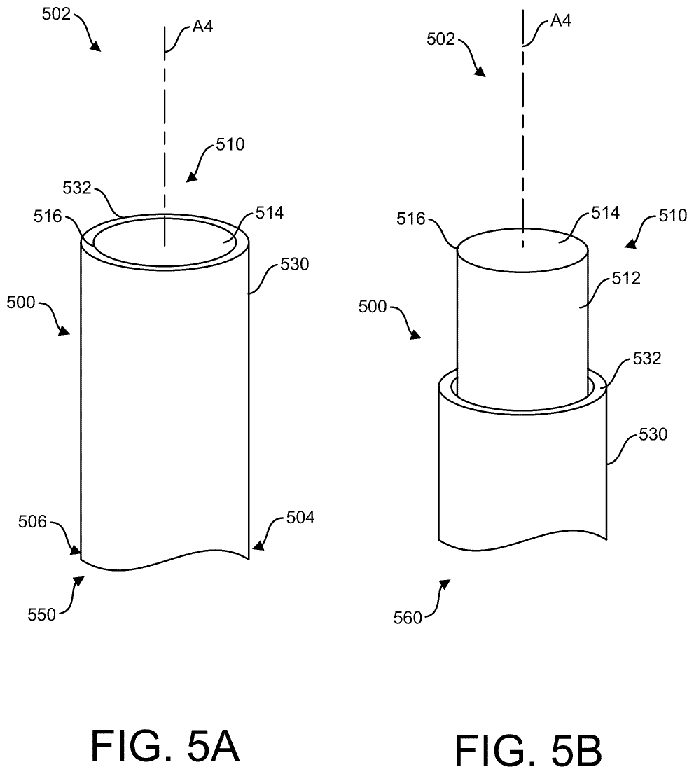

FIG. 5 illustrates still another example electrode assembly 500 of an example electrosurgical device 502, the electrosurgical device 502 constructed in accordance with the electrosurgical device 200. In the example, the electrode assembly is transitionable between a first, extended position 550, illustrated in FIG. 5A, to a second, retracted position 560, illustrated in FIG. 5B.

The electrosurgical device 502 includes a longitudinally extending shaft 504 having a distal region 506, and the shaft 504 defines a longitudinal axis A4 in the example. The electrode assembly 500 includes an electrode 510 disposed on the distal region 306. The electrode 510 is electrically coupled to the electrical connector 212 and the controller 214 via the electrical conductors within the shaft 504. The electrode 510 is constructed from a conductive material, such as metal, and suitable to carry an RF signal from electrosurgical unit 102. The electrode 510 includes a longitudinal surface 512 and a generally planar, exposed distalmost surface 514. In the example, the distalmost surface 514 is generally perpendicular to the axis A4, although other configurations such as an angled and planar exposed distalmost surface. The longitudinal surface 512 surrounds the electrode 510. The distalmost surface 514 defines a distal edge 516, such as the union or ridge where the distalmost surface 514 meets the longitudinal surface 512. In the example, the electrode 510 is cylindrical such that it includes one longitudinal surface 512 and one distal edge 516 that extends around the distalmost surface 514. Another example can include a rectangular electrode that includes four sub-longitudinal surfaces and the distal edge can comprise four sub-edges that extend around a square or rectangular distalmost surface. Still other examples are contemplated.

The electrode assembly 500 also includes a movable insulator 530 disposed on the electrode 510. The insulator 530 covers the longitudinal surface 512. In the example, the axially movable insulator 530 is transitionable between an extended position 500, illustrated in FIG. 5A, in which an insulator distal side 532 of the insulator 530 is flush with the edge 516 of the electrode 310 to a retracted position 560, illustrated in FIG. 5B, in which the insulator distal side 532 is retracted from the edge 516 to expose at least some, or a portion, of the longitudinal surface 512. The insulator 530 is constructed from a suitable insulative material and may be formed to slide between the first and second positions 550, 560. For example, in the first position, the insulator 530 surrounds the electrode 510 on the longitudinal surface 512, or sub-longitudinal surfaces, and includes the insulator distal side 532 that is generally flush with the distalmost surface 514. In the example first position 550, the insulator 530 extends distally all the way to the edge 516 such that the insulator distal side 532 coincides with the edge 516 and does not extend distally past the distalmost surface 514. In the example first position 550, no part of the longitudinal surface 512 of the electrode 510 is exposed, and all of the longitudinal surface 512 is covered with the insulator 530. When the electrode 510 is activated in the first position 550, only RF energy from the distalmost surface 514 can be applied to the patient. In the example second position 560, the insulator 530 is retracted to expose at least a portion of the longitudinal surface 512. When the electrode 510 is activated in the second position, RF energy from both the distalmost surface 514 and the longitudinal surface 512 can be applied to the patient.

In one example, the insulator 530 is transitionable between the first position 550 and the second position 560 via mechanical controls that slide the insulator 530 from the handle, such as handle 210 on a handheld electrosurgical device, such as electrosurgical device 200.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electrosurgical device, comprising:
a longitudinally extending shaft having a distal region, the shaft defining an axis;
an electrode disposed on the distal region, the electrode having a longitudinal surface and a generally planar, exposed distalmost surface, the distalmost surface defining an edge; and
an insulator disposed on the longitudinal surface to the edge, the insulator surrounding the electrode and flush with the distalmost surface.

2. The electrosurgical device of claim 1, wherein the insulator includes a distal side flush with the distalmost surface.

3. The electrosurgical device of claim 1, wherein the insulator comprises a heat shrink polytetrafluoroethylene.

4. The electrosurgical device of claim 1, wherein the electrode comprises nitinol.

5. The electrosurgical device of claim 1, wherein the shaft includes a proximal region, and further comprising a handle coupled to the proximal region.

6. The electrosurgical device of claim 1, wherein the shaft is one of flexible and generally rigid.

7. The electrosurgical device of claim 6, wherein the shaft is generally rigid and the electrosurgical device is a handheld device.

8. The electrosurgical device of claim 1, wherein the electrode is cylindrical.

9. The electrosurgical device of claim 1, wherein the electrode includes an outer puck disposed on a core mandrel.

10. The electrosurgical device of claim 9, wherein shaft carries an electrical conductor, and the core mandrel is electrically coupled to the electrical conductor.

11. The electrosurgical device of claim 10, wherein the core mandrel is integrally formed with the electrical conductor.

12. The electrosurgical device of claim 9, wherein the outer puck is threaded onto the core mandrel.

13. The electrosurgical device of claim 9, wherein the outer puck is welded onto the core mandrel.

14. The electrosurgical device of claim 1, wherein the shaft is configured as a guidewire.

15. An electrosurgical system, comprising:
an electrosurgical unit configured to provide a source of radiofrequency energy; and
an electrosurgical device operably coupled to the electrosurgical unit, the electrosurgical device comprising:
a longitudinally extending shaft having a distal region, the shaft defining an axis;
an electrode disposed on the distal region, the electrode configured to receive the source of radiofrequency energy from the electrosurgical unit, the electrode having a longitudinal surface and a generally planar, exposed distalmost surface, the distalmost surface defining an edge; and
an insulator disposed on the longitudinal surface to the edge, the insulator surrounding the electrode and flush with the distalmost surface.

16. The electrosurgical system of claim 15, wherein the electrosurgical device is configurable in a plurality of functions including a cut function and a coagulation function.

17. The electrosurgical system of claim 15, wherein the electrosurgical device is configurable in a monopolar mode.

18. The electrosurgical system of claim 15, and further comprising a pump couplable to source of fluid via delivery tubing, the electrosurgical unit configured to operate the pump, and the delivery tubing operably coupled to electrosurgical device.

19. An electrosurgical device, comprising:
a longitudinally extending shaft having a distal region, the shaft defining an axis;
an electrode disposed on the distal region, the electrode having a longitudinal surface and a generally planar, exposed distalmost surface, the distalmost surface defining an edge; and
an insulator disposed on the longitudinal surface to the edge, the insulator surrounding the electrode and flush with the distalmost surface;
wherein the insulator is transitionable to a retracted position wherein a portion of the longitudinal surface is exposed.

20. The electrosurgical device of claim 19, wherein the insulator is slidable along the longitudinal surface.
